(19) United States
(12) Patent Application Publication
(10) Pub. No.: US 2025/0009026 A1
(43) Pub. Date: Jan. 9, 2025

(54) VAPORISER DEVICE, CARTRIDGE, INHALER, AND METHOD FOR MANUFACTURING A VAPORISER DEVICE

(71) Applicant: KÖRBER TECHNOLOGIES GMBH, HAMBURG (DE)

(72) Inventors: Jan JAKLIN, EISLINGEN (DE); Lasse CORNILS, HAMBURG (DE); Niklas ROMMING, PLÖN (DE); Svenja KÄTHNER, HAMBURG (DE); Frank GOLDSCHMIDTBÖING, ORTENBERG (DE); Uwe PELZ, NAUENBURG (DE); Muhannad GHANAM, FREIBURG (DE)

(21) Appl. No.: 18/706,235

(22) PCT Filed: Nov. 7, 2022
(86) PCT No.: PCT/EP2022/080948

§ 371 Date: Apr. 30, 2024

(30) Foreign Application Priority Data
Nov. 10, 2021 (DE) 10 2021 129 264.6

(51) Int. Cl. A24F 40/46; A24F 40/10; A24F 40/42; A24F 40/70; A61M 15/00; H05B 3/03

(52) U.S. Cl. CPC A24F 40/46; A24F 40/10; A24F 40/42; A24F 40/70; A61M 15/0001; H05B 3/03

(57) ABSTRACT

A vaporizer device for an inhaler, preferably for an electronic cigarette product or a medical inhaler, comprising:
a vaporizer, for vaporizing liquid supplied to the vaporizer, in the form of an electrical resistance heating element made of doped silicon, and
at least one electrical line connected to the vaporizer, wherein
the at least one electrical line is electrically connected to the vaporizer by a layer sequence, wherein the layer sequence comprises the following layers which, starting from the vaporizer, follow one another:
a contact layer comprising an aluminum content, which is in contact with the vaporizer,
a diffusion barrier comprising a titanium content,
an adhesive layer comprising a nickel or titanium content, and
a connection layer comprising, for example, a silver or gold content, via which the layer sequence is electrically connected to the at least one line.

The present invention relates to a vaporizer device for an inhaler according to the preamble of claims 1, 9 and 13, and to a method for producing a vaporizer device. The invention further relates to a cartridge according to the preamble of claim 21 and to an inhaler according to the preamble of claim 22.

To vaporize liquids, it is generally known to use a resistance heater that heats up when an electrical heating voltage is applied. Usually, the liquid stored in a tank is supplied to the resistance heater through a wick structure so that it can be vaporized on the heated resistance heater or in the vicinity of the resistance heater.

In particular, the electrical connection of the resistance heater to an electrical voltage source is of crucial importance for reliable operation of an inhaler.

EP 2 316 286 A1 discloses an electrical cigarette having a resistance heater which comprises electrically conductive regions which are in turn applied on a non-conductive substrate. The resistance heater is connected to an electrical voltage source by means of a connection piece of the conductive region.

Furthermore, WO 2014/037794 A2 discloses an electrical cigarette product comprising one or more microheaters. The microheater has an electrically conductive layer which is embedded between a base layer and a protective layer. The microheater is connected to a voltage source by means of connection points of the electrically conductive layer which are not covered by the protective layer.

U.S. Pat. No. 5,573,692 also discloses a heater for an electrical cigarette product. A lower heating layer made of platinum is provided on a substrate and heats up when an electrical heating voltage is applied. For this purpose, a connection pin made of copper is connected to the heating layer via a contact layer made of platinum.

However, the disadvantage of these solutions is that they do not fulfill the desired properties, in particular with regard to the required service life and fail-safeness, in particular for use with a silicon vaporizer as a resistance heater.

In the case of electrical contacting of resistive silicon heaters as vaporizers in an electrical cigarette or in a medical inhaler, metallic contacts, so-called contact pads, are required. Requirements that do not arise for conventional applications of silicon components are placed on these contact pads. These requirements result from the operating conditions of an inhaler, which are influenced by the following aspects. First, the high operating temperatures of up to 300° C. and the large temperature fluctuations should be mentioned. In conventional solutions from the microelectronics sector, diffusion processes, which degrade the contact structure and thus unacceptably reduce the service life of the contacts, occur in this temperature range. Secondly, the wetting of the contact pad with liquid to be vaporized should also be mentioned, which can lead to failure of the contact pads, in particular if a typical liquid to be vaporized for an electrical cigarette wets the contact pad. In addition to water, such liquids typically also comprise polyethylene glycol, glycerol, nicotine and flavors.

Some metals, in particular the aluminum usually used for electrical contacting of silicon, corrode upon contact with such liquids, which can lead to failure of conventional contact pads.

The use of gold instead of aluminum is also disadvantageous for electrical contacting of silicon. However, gold does not dissolve the oxide on the silicon surface, so the oxide must be removed wet-chemically shortly before applying the gold. A pure gold contact also leads to the problem that, at the interface between gold and silicon, silicon atoms are released permanently—and more strongly at elevated temperature—from the crystal lattice and then diffuse through the gold. At the surface of the contact pad made of gold, these silicon atoms oxidize and form a vitreous layer. Due to the instability of the gold-silicon interface and the oxide-induced adhesion problems of gold on silicon, gold contacts are not used commercially. Furthermore, the resulting vitreous layer at the contact pads made of gold prevents the connection to an electrical line by means of silver sintering.

So-called bonding metals or alloys such as titanium, chromium, or tungsten-titanium are suitable for forming a largely stable interface with silicon. However, this takes place with the formation of a so-called Schottky contact, which, however, is only properly conductive for one current direction and is therefore unsuitable for contacting a resistance heater.

The object of the invention is to provide a vaporizer device comprising a vaporizer made of doped silicon with improved system reliability properties, and to provide a correspondingly improved cartridge, a correspondingly improved inhaler, and a corresponding method for producing a vaporizer device.

The object is achieved by the features of the independent claims. Further preferred embodiments of the invention can be found in the dependent claims, the figures, and the associated description.

Some terms used within the scope of this application are explained first.

Within the context of this application, an electrical line is to be understood as any conductive material which is suitable for applying an electrical heating voltage to a vaporizer, i.e., for establishing an electrical connection to a voltage source.

A layer comprising a content of a certain element, material or alloy is to be understood as any detectable content up to a theoretical content of 100%.

According to a first aspect of the application, the object is achieved by a vaporizer device for an inhaler, preferably for an electronic cigarette product or a medical inhaler, comprising a vaporizer, for vaporizing liquid supplied to the vaporizer, in the form of an electrical resistance heating element made of doped silicon, and at least one electrical line connected to the vaporizer, wherein the at least one electrical line is electrically connected to the vaporizer by a layer sequence, wherein the layer sequence comprises the following layers which, starting from the vaporizer, follow one another: a contact layer comprising an aluminum content, which is in contact with the vaporizer; a diffusion barrier comprising a titanium content; an adhesive layer comprising a nickel or titanium content; and a connection layer comprising, for example, a silver or gold content, via which the layer sequence is electrically connected to the at least one line.

The invention is based on the finding that the vaporizer can be connected to the at least one electrical line particularly efficiently and stably by the proposed layer sequence.

The aluminum of the contact layer produces an efficient contact of the layer sequence to the vaporizer made of silicon. The titanium content of the diffusion barrier prevents diffusion of the aluminum toward the surface, thereby increasing the longevity of the vaporizer device. Furthermore, by means of the diffusion barrier, the silicon-metal transition region is separated from a metal-metal layer which arises during connection to the electrical line; undesired or functionally critical reactions between these two regions thus cannot occur, either during production or during operation.

It has been found that the adhesive layer with a nickel or titanium content allows a reliable connection of the connection layer. The connection layer forms a protective layer for the underlying layers of the layer sequence, so that the contact pad formed by the proposed layer sequence is protected from corrosion. Furthermore, the connection layer causes the underlying layers not to negatively affect or contaminate the vaporized liquid.

It has been shown in tests that the proposed layer structure allows a service life of at least 500 heating cycles up to a temperature of 300° C. over a heating duration of 3 seconds in each case. In particular, the boundary layer to the silicon has proven to be stable. This also applies if contact pads in the form of the proposed layer sequence are dipped into a liquid to be vaporized, for example into the liquid for an electrical cigarette. The vaporizer device thus has excellent system reliability properties. Furthermore, it has been shown that the layer sequence makes it possible to produce an ohmic contact between the layer sequence and the vaporizer made of silicon, so that an efficient electrical connection of the vaporizer to a voltage source is made possible.

Furthermore, the proposed layer sequence can be applied to the vaporizer with low manufacturing effort, so that the production costs can be reduced.

Preferably, the connection layer is a layer made of noble metal, for example silver or gold. In principle, however, it is also possible for the connection layer to be formed from a non-noble metal if the connection layer electrically connected to the line is shut off from the surroundings by an encapsulation.

Preferably, the vaporizer is formed from p-doped silicon.

Preferably, the surface of the contact layer that is not covered by the vaporizer or by the diffusion barrier is covered by a passivation layer. Preferably, the surface of the contact layer covered by the passivation layer is a side surface. In this way, the contact layer, which is particularly susceptible to corrosion, can be protected from corrosive influences. For example, the passivation layer comprises a content of silicon dioxide, a content of silicon nitride or a content of silicon carbide.

The passivation layer preferably comprises at least 80 wt. % of silicon dioxide, at least 80 wt. % of silicon nitride or at least 80 wt. % of silicon carbide. It has been shown that these material concentrations are particularly suitable for passivation of the contact layer. More preferably, the content of silicon dioxide, silicon nitride, or silicon carbide is at least 90 wt. %, in particular preferably at least 95 wt. %.

It is further preferred if the passivation layer is overlapped by at least one further layer, for example by the connection layer. The connection layer or another layer preferably overlaps the passivation layer by at least 1 μm, so that the contact surface between the passivation layer and the connection layer or the other layer is reliably hermetically sealed. If the passivation layer is overlapped by the connection layer, an encapsulation of the contact layer, the adhesive layer, and the diffusion layer on the vaporizer can be achieved by the connection layer together with the passivation layer.

It is preferred if the contact layer comprises at least 80 wt. % of aluminum or at least 80 wt. % of aluminum-silicon-copper. More preferably, the content of aluminum or aluminum-silicon-copper is at least 90 wt. %, in particular preferably at least 95 wt. %. The contact layer preferably has a layer thickness between 0.5 μm and 3.5 μm, more preferably between 1.5 μm and 3.0 μm, in particular preferably 2.5 μm. This formation of the contact layer has proven to be advantageous for connecting the layer sequence to the vaporizer with simultaneously high electrical conductivity. Aluminum-silicon-copper as a contact layer offers the advantage that it is not very susceptible to diffusion processes.

It is preferred if the diffusion barrier comprises at least 80 wt. % of titanium or at least 80 wt. % of titanium nitride. More preferably, the content of titanium or titanium nitride is at least 90 wt. %, in particular preferably at least 95 wt. %. More preferably, the diffusion barrier has a layer thickness between 25 nm and 100 nm, more preferably between 40 nm and 60 nm, in particular preferably 50 nm. It has been shown that a diffusion barrier designed in this way allows effective protection against diffusion of the aluminum atoms from the contact layer with simultaneously high electrical conductivity.

By using titanium or titanium nitride as a diffusion barrier, diffusion of the aluminum from the contact layer toward the surface can be prevented even at higher temperatures.

It is preferred if the adhesive layer comprises at least 80 wt. % of nickel, at least 80 wt. % of nickel-vanadium, or at least 80 wt. % of titanium nitride. More preferably, the content of nickel, nickel-vanadium, or titanium nitride is at least 90 wt. %, in particular preferably at least 95 wt. %. The nickel-based adhesive layer preferably has a layer thickness between 50 nm and 150 nm, preferably between 80 nm and 120 nm, in particular preferably 100 nm. It has been shown that an adhesive layer designed in this way allows a particularly good adhesive effect with simultaneously high electrical conductivity.

The silver content or the gold content in the connection layer is preferably at least 80 wt. %, more preferably at least 90 wt. %, in particular preferably 95 wt. %. The connection layer preferably has a layer thickness between 125 nm and 375 nm, more preferably between 200 nm and 300 nm, in particular preferably 250 nm. By means of such a connection layer with a noble metal, the underlying layers can be protected from corrosive influences. Furthermore, the user can be protected from negative influences of the underlying layers by the protective layer made of noble metal. Furthermore, the connection layer made of silver or of the silver alloy offers the advantage that it can be contacted particularly well by means of silver sintering; a connection to the electrical line with particularly good electrical and mechanical properties is thus possible, because the connection layer and the sinter paste material are identical and therefore fewer impurities and lower mechanical or thermal stresses occur. The silver layer formed by the silver sintering can be understood here as a component of the electrical line.

It has further been shown that in particular the combination of nickel-vanadium as the adhesive layer and silver as the connection layer exhibits high mechanical heavy load values.

According to a second aspect of the application, the object is achieved by a vaporizer device for an inhaler, preferably for an electronic cigarette product or a medical inhaler, comprising a vaporizer, for vaporizing liquid supplied to the vaporizer, in the form of an electrical resistance heating element made of doped silicon, and at least one electrical line connected to the vaporizer, wherein the at least one line is electrically connected to the vaporizer by a layer sequence, wherein the layer sequence, starting from the vaporizer, comprises the following layers: a contact layer comprising a gold content, which is in contact with the vaporizer; and a diffusion barrier comprising a platinum content.

The gold content of the contact layer makes it possible to produce a sufficiently high adhesive strength to the vaporizer made of silicon, preferably of p-doped silicon. Due to the diffusion barrier arranged above it with the platinum content, the silicon atoms released at the interface between gold and silicon cannot penetrate through to the outside. Furthermore, the diffusion barrier with the platinum content offers the advantage that it forms a stable boundary layer to the contact layer underneath it, comprising the gold content. High corrosion resistance can thus also be achieved when the layer sequence is in direct contact with a liquid to be vaporized of an electrical cigarette. Furthermore, by means of the diffusion barrier, the silicon-metal transition region is separated from a metal-metal layer which arises during connection to the electrical line; undesired reactions between these two regions thus cannot occur, either during production or during operation.

The connection of the electrical line can in principle take place directly at the diffusion barrier, for example by silver sintering on the diffusion barrier made of platinum. The layer sequence can thus be connected simply to the electrical line by silver sintering. The silver layer formed by the silver sintering can be understood here as a component of the electrical line.

The proposed layer structure allows a service life of at least 500 heating cycles up to a temperature of 300° C. over a heating duration of 3 seconds in each case. In particular, the boundary layer to the silicon has proven to be reliable. This also applies if contact pads in the form of the layer sequence are dipped into a liquid to be vaporized, for example into the liquid for an electrical cigarette. The vaporizer device thus has excellent system reliability properties. Furthermore, it has been shown that the layer sequence makes it possible to produce an ohmic contact between the layer sequence and the vaporizer made of silicon, so that an efficient electrical connection of the vaporizer to a voltage source is made possible.

Furthermore, the proposed layer sequence can be applied to the vaporizer with low manufacturing effort, so that the production costs can be reduced.

The contact layer preferably comprises a gold content of at least 80 wt. % and/or the diffusion barrier comprises a platinum content of at least 80 wt. %. More preferably, the gold content in the contact layer and/or the platinum content in the diffusion barrier is at least 90 wt. %, in particular preferably at least 95 wt. %. This is advantageous because the platinum can be deposited on the contact layer made of gold in a simple manner.

It is preferred if the layer sequence has a connection layer comprising a silver or gold content, wherein the connection layer is arranged on the diffusion barrier, wherein the layer sequence is electrically connected to the line via the connection layer. The silver or gold content in the connection layer is preferably at least 80 wt. %, more preferably at least 90 wt. %, in particular preferably at least 95 wt. %. By means of the additional connection layer, the connection of the electrical line by means of silver sintering can be simplified, because silver sintering is possible on materials comprising a gold or silver content with less manufacturing effort.

As an alternative to the connection layer with a gold or silver content, it is in principle also possible for the connection layer to be formed from a non-noble metal if the connection layer electrically connected to the line is shut off from the surroundings by an encapsulation.

Preferably, the contact layer has a layer thickness between 25 nm and 100 nm, more preferably between 40 nm and 60 nm, in particular preferably 50 nm. The diffusion barrier preferably has a layer thickness between 50 nm and 150 nm, more preferably between 80 nm and 120 nm, in particular preferably 100 nm. Preferably, the connection layer has a layer thickness between 125 nm and 400 nm, more preferably between 200 nm and 300 nm, in particular preferably 250 nm. These layer thicknesses have proven to be advantageous for good electrical contacting with simultaneously high corrosion resistance.

According to a third aspect of the application, the object is achieved by a vaporizer device for an inhaler, preferably for an electronic cigarette product or a medical inhaler, comprising a vaporizer, for vaporizing liquid supplied to the vaporizer, in the form of an electrical resistance heating element made of doped silicon, and at least one electrical line connected to the vaporizer, wherein the at least one line is electrically connected to the vaporizer by a layer sequence, wherein the layer sequence, starting from the vaporizer, comprises the following layers: an adhesive layer comprising, for example, a titanium content; and a connection layer comprising a gold, silver or platinum content, via which the layer sequence is electrically connected to the line; wherein the vaporizer has locally, directly adjacent to the adhesive layer, a region of higher dopant concentration than in the remaining part of the vaporizer.

The region of high dopant concentration can arise, for example, by diffusion from a boron trichloride source. Alternatively, however, doping with other dopants or doping sources is also possible.

Due to the locally high dopant concentration, a high charge carrier concentration forms at the vaporizer near the surface, so that contacting with conventional bonding metals, for example with titanium, chromium or tungsten-titanium, is made possible.

The region of high dopant concentration preferably extends over the entire surface of the upper side of the vaporizer or over the entire surface of the vaporizer.

Preferably, the vaporizer is formed from p-doped silicon.

In the region of higher dopant concentration, the dopant concentration is preferably at least 3*10¹⁹ 1/cm3, for example at least 5*10¹⁹ 1/cm3. The region of higher dopant concentration preferably projects into the vaporizer between 20 nm and 1000 nm deep, for example between 50 nm and 500 nm deep, in particular 200 nm deep. By means of such a design of the region of high dopant concentration, sufficiently good contacting can be achieved without the heating properties of the rest of the vaporizer, which usually has a layer thickness of approx. 300 μm, being impaired. Due to the low penetration depth into the vaporizer, the region of high dopant concentration is likewise layered, so that it can be regarded as a component of the layer sequence.

It has also proven to be advantageous if the adhesive layer comprises a titanium content of at least 80 wt. %, more preferably of at least 90 wt. %, in particular preferably of at least 95 wt. %.

A diffusion barrier comprising a platinum content is preferably arranged between the adhesive layer and the connection layer. The diffusion barrier makes it possible to prevent the diffusion of silicon atoms into the surroundings, as a result of which the service life of the vaporizer device can be increased. Furthermore, by means of the diffusion barrier, the silicon-metal transition region is separated from a metal-metal layer which arises during connection to the electrical line; undesired reactions between these two regions thus cannot occur, either during production or during operation. The diffusion barrier comprises a platinum content of at least 80 wt. %, more preferably of at least 90 wt. %, and in particular preferably of at least 95 wt. %. It has been shown that in particular the adhesive layers with a titanium content in combination with the diffusion barrier comprising a platinum content is advantageous, because a structure of the layer sequence which is stable over a plurality of heating cycles is thereby made possible.

The connection layer preferably comprises a silver, gold or platinum content of at least 80 wt. %, more preferably at least 90 wt. %, in particular preferably at least 95 wt. %. Such a connection layer allows the electrical connection of the line by means of silver sintering, i.e., with low manufacturing effort.

For the vaporizer device according to the first, second and third aspects of this application, the layers of the layer sequence preferably follow one another directly. There are thus no intermediate layers, so that the layers of the layer sequence can interact ideally.

For the vaporizer device according to the first, second and third aspects of this application, preferably at least two of the layer sequences are provided, wherein these are arranged at the surface of the vaporizer locally at a distance from one another. Each layer sequence is then connected to an electrical line. If exactly two layer sequences are applied to the vaporizer, a first layer sequence can be connected to a positive pole of a voltage source by a first electrical line, and a second layer sequence can be connected to a negative pole of the voltage source by a second electrical line.

According to a fourth aspect of the application, the object is achieved by a method for producing a vaporizer device according to the first, second or third aspect of this application, wherein, in a first method step a), the layers of the layer sequence are deposited on the vaporizer; and, in a subsequent second method step b), a temperature control of the layer sequence to a temperature between 400° C. to 600° C., for example between 450° C. and 550° C., takes place over a predefined time period. Furthermore, for example, the temperature in method step b) is approx. 500° C. By means of method step b), the internal stresses of the layer sequence that can arise after method step a) can be reduced. If these internal stresses in the layer sequence were not eliminated, delamination effects or the formation of fine cracks would occur, in particular due to the cyclic thermal loading during operation of the vaporizer device. By controlling the temperature in method step b), these effects can be prevented and the corrosion resistance can thus be improved.

According to a fifth aspect of the application, the object is achieved by a cartridge for an inhaler, preferably for an electronic cigarette product or a medical inhaler, comprising a liquid tank for storing a liquid to be vaporized, wherein the cartridge comprises a vaporizer device according to the first, second or third aspect of this application. With regard to the technical effects and advantages associated with the cartridge, reference is made to the above statements in connection with the vaporizer device according to the first, second or third aspect of this application.

According to a sixth aspect of the application, the object is achieved by an inhaler, in particular in the form of an electronic cigarette product or a medical inhaler, comprising a flow channel, a liquid tank for storing liquid to be vaporized, and a voltage source, wherein the inhaler comprises a vaporizer device according to a first, second or third aspect of this application, wherein the at least one electrical line of the vaporizer device is connected to the voltage source so that an electrical heating voltage can be applied to the vaporizer. With regard to the technical effects and advantages associated with the inhaler, reference is made to the above statements in connection with the vaporizer device according to the first, second or third aspect of the application.

The invention is explained below using preferred embodiments with reference to accompanying figures. Shown are:

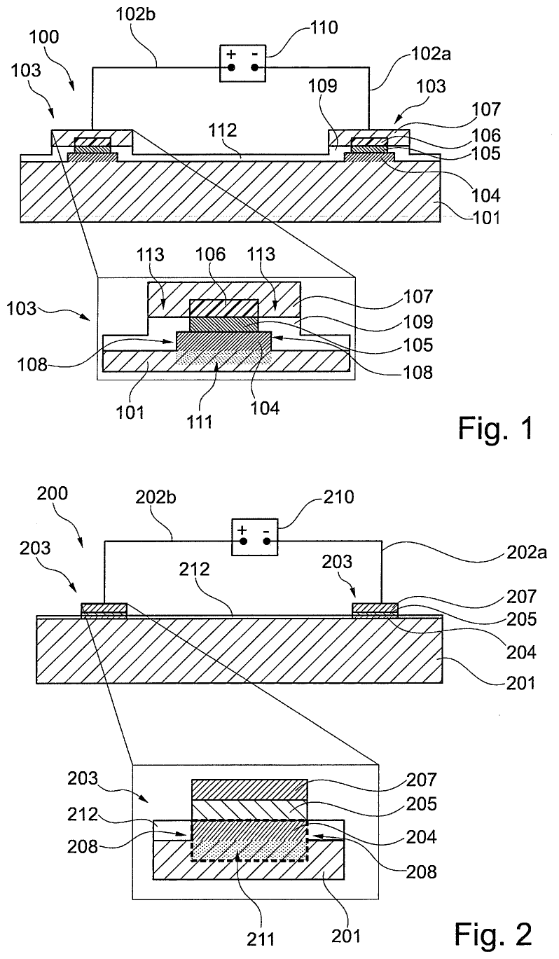

FIG. 1 a first embodiment of a vaporizer;

FIG. 2 a second embodiment of a vaporizer;

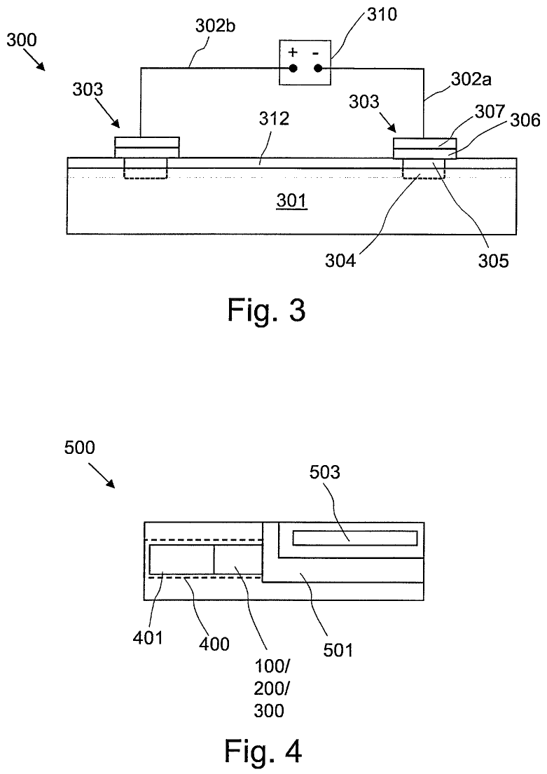

FIG. 3 a third embodiment of a vaporizer;

FIG. 4 an inhaler; and

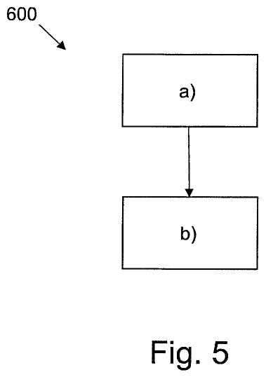

FIG. 5 a schematic representation of a method for producing a vaporizer according to the first embodiment.

FIG. 1 shows a vaporizer device 100 according to a first embodiment, comprising a vaporizer 101 which is connected to two electrical lines 102a, 102b via two layer sequences 103. The electrical lines 102a, 102b are in turn connected to a voltage source 110. In this case, the line 102a is connected to the negative pole and the line 102b is connected to the positive pole of the voltage source 110. The voltage source 110 can be, for example, an energy store in the form of a battery or a rechargeable battery.

The vaporizer 101 is a resistance heater made of p-doped silicon, so that it heats up when a heating voltage is applied.

In order to avoid repetitions, only the structure of one of the two layer sequences 103 is explained below. However, the two layer sequences 103 shown have an identical structure. The layer sequence 103 is explained below on the basis of the enlarged representation of the left layer sequence 103 shown at the bottom of FIG. 1.

Starting from the vaporizer 101, the layer sequence 103 comprises a contact layer 104 made of aluminum-silicon-copper with a layer thickness of 2.5 μm. In principle, however, the layer thickness can also be between 0.5 μm and 3.5 μm, for example. It can be seen that the contact layer 104 transitions into the vaporizer 101 in a transition region 111, i.e., no sharp separation of the vaporizer 101 from the contact layer 104 is possible. The layer thickness is therefore determined starting from the adjacent surface of the vaporizer 101. Alternatively, the contact layer 104 can also be formed from pure aluminum, i.e., with a degree of purity of more than 99 percent by weight.

A diffusion barrier 105 made of titanium with a layer thickness of 50 nm is applied to the contact layer 104. In principle, the layer thickness can also be between 25 and 100 nm, for example. As an alternative to titanium, the diffusion barrier 105 can also be formed by titanium nitride.

By means of an adhesive layer 106 applied to the diffusion barrier 105, the connection layer 107 can be reliably connected to the remaining layers of the layer sequence 103.

The adhesive layer 106 is formed from nickel and has a layer thickness of 100 nm. In principle, the layer thickness can also be between 50 nm and 150 nm, for example. As an alternative to pure nickel, the adhesive layer 106 can also be formed by nickel-vanadium, for example.

The connection layer 107 is formed from silver and has a layer thickness of 250 nm. In principle, the connection layer 107 can also be formed by a silver alloy. Electrical contacting with the line 102b can take place by silver sintering, because the connection layer 107 comprises a silver content. The layer produced by silver sintering is to be regarded as a component of the line 102b. In principle, a layer thickness of the connection layer between 125 nm and 375 nm is also possible, for example.

If the respective layers of the layer sequence 103 do not have a constant layer thickness, the layer thickness is determined starting from the maximum layer thickness. This applies to all embodiments.

It can also be seen from FIG. 1 that the contact layer 104 is covered by a passivation layer 109 on an otherwise exposed surface 108, here a side surface. This passivation layer 109 does not necessarily have to extend over the entire surface of the vaporizer 101, but can transition into an oxide layer 112 which forms anyway on the surface of the vaporizer 101. The contact layer 104, the diffusion barrier 105 and the adhesive layer 106 are encapsulated, i.e., hermetically sealed with respect to the surroundings, by the passivation layer 109 and the connection layer 107, so that the layers encapsulated therein are protected from corrosive influences. In this exemplary embodiment, the passivation layer 109 is formed from silicon dioxide. Alternatively, the passivation layer 109 can also be formed by silicon nitride or silicon carbide, for example. It can also be seen that the passivation layer 109 is overlapped at least 1 μm by the connection layer 107. A hermetic seal of the inner layers with respect to the surroundings can be achieved in a particularly reliable manner by an overlapping region 113 created in this way. Protection from corrosive influences can thus be achieved and at the same time emission of potentially harmful substances can be prevented.

By means of the proposed layer sequence 103, a reliable and also good electrically-conductive connection of the vaporizer 101 to the voltage source 110 can take place by means of electrical lines 102a and 102b.

The same effect can also be achieved with a contact layer 104 made of aluminum or aluminum-silicon-copper, a diffusion barrier 105 made of titanium, an adhesive layer 106 made of titanium nitride, and a connection layer 107 made of gold.

FIG. 2 shows a vaporizer device 200 according to a second embodiment, comprising a vaporizer 201 which is connected to an electrical line 202a, 202b by two layer sequences 203. The electrical lines 202a, 202b are in turn connected to a voltage source 210. In this case, the line 202a is connected to the negative pole and the line 202b is connected to the positive pole of the voltage source 210. The voltage source 210 can be, for example, an energy store in the form of a battery or a rechargeable battery.

The vaporizer 201 is a resistance heater made of p-doped silicon, so that it heats up when a heating voltage is applied.

In order to avoid repetitions, only the structure of one of the two layer sequences 203 is explained below. However, the two layer sequences 203 shown have an identical structure. The layer sequence 203 is explained below on the basis of the enlarged representation of the left layer sequence 203 shown at the bottom of FIG. 2.

Starting from the vaporizer 201, the layer sequence 203 comprises a contact layer 204, a diffusion barrier 205, and a connection layer 207.

The contact layer 204 made of gold has a layer thickness of 50 nm. In principle, however, layer thicknesses between 25 nm and 100 nm are possible. It can be seen that the contact layer 204 transitions into the vaporizer 201 in a transition region 211, i.e., no sharp separation of the vaporizer 201 from the contact layer 204 is possible. The layer thickness is therefore determined starting from the adjacent surface of the vaporizer 101. Adjacent to the contact layer 204, a natural oxide layer 212 forms on the surface of the vaporizer 201, which oxide layer covers an otherwise exposed surface 208 of the contact layer 204 that is not covered by the diffusion barrier 205.

A diffusion barrier 205 made of platinum with a layer thickness of 100 nm follows the contact layer 204. In principle, however, layer thicknesses of, for example, between 50 nm and 150 nm are also possible.

A connection layer 207 made of gold with a layer thickness of 250 nm follows the diffusion barrier 205. In principle, however, the connection layer 207 can also have a layer thickness between 125 nm and 400 nm, for example. Furthermore, the connection layer 207 can also be formed by silver as an alternative to gold. An electrical connection to the electrical line 202b can be carried out via the connection layer 207 by silver sintering. The layer formed by silver sintering on the connection layer 207 can thereby be regarded as a component of the line 202b. Furthermore, a variant is also conceivable in which the connection layer 207 is dispensed with. The connection of the electrical line 203b then takes place via the diffusion barrier 205 made of platinum.

In this way, a reliable electrical contacting of the vaporizer 201 can take place, so that it can be connected to the voltage source 210 by the layer sequence 203 and the electrical lines 202a and 202b. An electrical heating voltage can thus be applied to the vaporizer 201, so that it heats up to vaporize a liquid. Since the layer sequence 203 only comprises layers of noble metals, it can be assumed to be safe for the human body.

FIG. 3 shows a third embodiment of a vaporizer device 300 comprising a vaporizer 301 which is connected to electrical lines 302a, 302b by two layer sequences 303. The electrical lines 302a, 302b are in turn connected to a voltage source 310. In this case, the line 302a is connected to the negative pole and the line 302b is connected to the positive pole of the voltage source 310. The voltage source 310 can be, for example, an energy store in the form of a battery or a rechargeable battery.

The vaporizer 301 is a resistance heater made of p-doped silicon, so that it heats up when a heating voltage is applied.

In order to avoid repetitions, only the structure of one of the two layer sequences 303 is explained below. However, the two layer sequences 303 shown have an identical structure. The layer sequence 303 is explained below on the basis of the layer sequence 303 arranged on the right in FIG. 3.

The vaporizer 301 comprises locally a region of higher dopant concentration 304, i.e., the dopant concentration is higher in this region than in the remaining region of the vaporizer 301. A first layer of the layer sequence 303 can thus be created by local doping of the vaporizer 301. The local doping can be generated, for example, by diffusion from a boron trichloride source. In this case, the region of higher dopant concentration 304 projects approximately 200 nm into the vaporizer 301. In this case, the vaporizer 301 itself has a layer thickness of approximately 300 μm. The region of higher dopant concentration 304 has a dopant concentration of more than 5*10¹⁹ 1/cm3.

An adhesive layer 305 made of titanium followed by a diffusion barrier 306 made of platinum follows the region of higher dopant concentration 304. In principle, however, the adhesive layer 305 can also be formed from other materials. Furthermore, it is in principle also conceivable for the diffusion barrier 306 to be completely omitted.

A natural oxide layer 312 forms adjacent to the adhesive layer 305 on the surface of the vaporizer 301.

A connection layer 307, which is formed from gold in this exemplary embodiment, follows the diffusion barrier 306, wherein a connection layer 307 made of silver or platinum is also conceivable in principle.

This layer sequence 303 allows reliable electrical contacting of the vaporizer 301. The vaporizer 301 is connected to the voltage source 310 by the layer sequence 303 and the electrical lines 302a and 302b. An electrical heating voltage can thus be applied to the vaporizer 301, so that it heats up to vaporize a liquid.

FIG. 4 shows an inhaler 500 comprising a flow channel 501 and a cartridge 400 in the form of a vaporizer-tank unit. The cartridge 400 comprises a vaporizer device 100, 200 or 300 according to the first embodiment (cf. FIG. 1), according to the second embodiment (cf. FIG. 2) or the third embodiment (cf. FIG. 3).

The cartridge 400 comprises a liquid tank 401 filled with a liquid to be vaporized. During operation of the vaporizer device 100, 200 or 300, i.e., when a heating voltage is applied to the vaporizer 101, 201 or 301, the liquid to be vaporized is heated up to the boiling point and thus emitted to the flow channel 501 in the vapor state. Furthermore, a voltage source 503 is shown as a component of the inhaler 500, by which voltage source the vaporizer device 100, 200 or 300 can be supplied with electrical energy.

FIG. 5 schematically shows a method 600 for producing the vaporizer device 100 according to the first exemplary embodiment shown in FIG. 1. In a first method step a), the layers of the layer sequence 103 are deposited on the vaporizer 101, i.e., the contact layer 104, the diffusion barrier 105, the adhesive layer 106 and the connection layer 107. After this layer sequence 103 has been formed, internal mechanical stresses can occur which can negatively affect the service life of the layer sequence 103. Therefore, in a second method step b), a temperature control of the layer sequence 103 takes place to a temperature between 400° C. to 600° C., preferably between 450° C. and 550° C., over a predefined time period. By means of this heating, the internal stresses can be almost completely eliminated, so that the delamination of individual layers or a plurality of layers can be prevented. In principle, however, the method 600 can also be applied to the vaporizer device according to the second embodiment (cf. FIG. 2) or the third embodiment (cf. FIG. 3).

1. A vaporizer device for an inhaler wherein said vaporizer device comprises:
a vaporizer, for vaporizing liquid supplied to the vaporizer, in the form of an electrical resistance heating element made of doped silicon, and
at least one electrical line connected to the vaporizer, wherein
the at least one electrical line is electrically connected to the vaporizer by a layer sequence, wherein the layer sequence comprises the following layers which, starting from the vaporizer, follow one another:
a contact layer comprising an aluminum content, which is in contact with the vaporizer,
a diffusion barrier comprising a titanium content,
an adhesive layer comprising a nickel or titanium content, and
a connection layer, via which the layer sequence is electrically connected to the at least one line.

2. The vaporizer device according to claim 1, wherein
a surface of the contact layer that is not covered by the vaporizer or by the diffusion barrier is covered by a passivation layer.

3. The vaporizer device according to claim 2, wherein
the passivation layer comprises at least 80 wt. % of silicon dioxide, at least 80 wt. % of silicon nitride or at least 80 wt. % of silicon carbide.

4. The vaporizer device according to claim 2, wherein
the passivation layer is overlapped by at least one further layer.

5. The vaporizer device according to claim 1, wherein
the contact layer comprises at least 80 wt. % of aluminum or at least 80 wt. % of aluminum-silicon-copper.

6. The vaporizer device according to claim 1, wherein
the diffusion barrier comprises at least 80 wt. % of titanium or at least 80 wt. % of titanium nitride.

7. The vaporizer device according to claim 1, wherein
the adhesive layer comprises at least 80 wt. % of nickel, at least 80 wt. % of nickel-vanadium or at least 80 wt. % of titanium nitride.

8. The vaporizer device according to claim 1, wherein the connection layer comprises a silver or gold content of at least 80 wt. %.

9. A vaporizer device for an inhaler wherein said vaporizer device comprises:
a vaporizer, for vaporizing liquid supplied to the vaporizer, in the form of an electrical resistance heating element made of doped silicon, and
at least one electrical line connected to the vaporizer; wherein
the at least one line is electrically connected to the vaporizer by a layer sequence, wherein the layer sequence, starting from the vaporizer, comprises the following layers:
a contact layer comprising a gold content, which is in contact with the vaporizer, and
a diffusion barrier comprising a platinum content.

10. The vaporizer device according to claim 9, wherein the contact layer comprises a gold content of at least 80 wt. % and/or the diffusion barrier comprises a platinum content of at least 80 wt. %.

11. The vaporizer device according to claim 9, wherein
the layer sequence has a connection layer comprising a silver or gold content, wherein
the connection layer is arranged on the diffusion barrier, wherein
the layer sequence is electrically connected to the line by the connection layer.

12. The vaporizer device according to claim 11, wherein
the connection layer comprises a silver or gold content of at least 80 wt. %.

13. A vaporizer device for an inhaler wherein said vaporizer device comprises:
a vaporizer, for vaporizing liquid supplied to the vaporizer, in the form of an electrical resistance heating element made of doped silicon, and
at least one electrical line connected to the vaporizer, wherein
the at least one line is electrically connected to the vaporizer by a layer sequence, wherein
the layer sequence, starting from the vaporizer, comprises the following layers:
an adhesive layer comprising, and
a connection layer comprising a gold, silver or platinum content, via which the layer sequence is electrically connected to the line, wherein
the vaporizer has locally, directly adjacent to the adhesive layer, a region of higher dopant concentration than in the remaining part of the vaporizer.

14. The vaporizer device according to claim 13, wherein
in the region of higher dopant concentration, the dopant concentration is at least 3*10¹⁹ 1/cm³.

15. The vaporizer device according to claim 13, wherein
the region of higher dopant concentration projects into the vaporizer between 20 nm and 1000 nm deep.

16. The vaporizer device according to claim 13, wherein the adhesive layer comprises a titanium content of at least 80 wt. %.

17. The vaporizer device according to claim 13, wherein
a diffusion barrier comprising a platinum content is arranged between the adhesive layer and the connection layer.

18. The vaporizer device according to claim 17, wherein
the diffusion barrier comprises a platinum content of at least 80 wt. %.

19. The vaporizer device according to claim 13, wherein
the connection layer comprises a silver, gold or platinum content of at least 80 wt. %.

20. A method for producing a vaporizer device according to claim 1, wherein
in a first method step a), the layers of the layer sequence are deposited on the vaporizer, and
in a subsequent second method step b), a temperature control of the layer sequence to a temperature between 400° C. to 600° C., takes place over a predefined time period.

21. A cartridge for an inhaler comprising a liquid tank for storing a liquid to be vaporized, wherein the cartridge comprises:
A) a vaporizer device according to claim 1; or
B) a vaporizer device that comprises:
a vaporizer, for vaporizing liquid supplied to the vaporizer, in the form of an electrical resistance heating element made of doped silicon, and
at least one electrical line connected to the vaporizer wherein
the at least one line is electrically connected to the vaporizer by a layer sequence, wherein the layer sequence, starting from the vaporizer, comprises the following layers:
a contact layer comprising a gold content, which is in contact with the vaporizer, and
a diffusion barrier comprising a platinum content: or
C) a vaporizer device that comprises:
a vaporizer, for vaporizing liquid supplied to the vaporizer, in the form of an electrical resistance heating element made of doped silicon, and
at least one electrical line connected to the vaporizer, wherein
the at least one line is electrically connected to the vaporizer by a layer sequence, wherein
the layer sequence, starting from the vaporizer, comprises the following layers:
an adhesive layer comprising, and
a connection layer comprising a gold, silver or platinum content, via which the layer sequence is electrically connected to the line, wherein
the vaporizer has locally, directly adjacent to the adhesive layer, a region of higher dopant concentration than in the remaining part of the vaporizer.

22. An inhaler comprising:
a flow channel,
a liquid tank for storing liquid to be vaporized, and
a voltage source, wherein
the inhaler comprises:
A) a vaporizer device according to claim 1; or
B) a vaporizer device that comprises:
a vaporizer, for vaporizing liquid supplied to the vaporizer, in the form of an electrical resistance heating element made of doped silicon, and
at least one electrical line connected to the vaporizer wherein
the at least one line is electrically connected to the vaporizer by a layer sequence, wherein the layer sequence, starting from the vaporizer, comprises the following layers:
a contact layer comprising a gold content, which is in contact with the vaporizer, and
a diffusion barrier comprising a platinum content; or
C) a vaporizer device that comprises:
a vaporizer, for vaporizing liquid supplied to the vaporizer, in the form of an electrical resistance heating element made of doped silicon, and
at least one electrical line connected to the vaporizer, wherein
the at least one line is electrically connected to the vaporizer by a layer sequence, wherein
the layer sequence, starting from the vaporizer, comprises the following layers:
an adhesive layer comprising, and
a connection layer comprising a gold, silver or platinum content, via which the layer sequence is electrically connected to the line, wherein
the vaporizer has locally, directly adjacent to the adhesive layer, a region of higher dopant concentration than in the remaining part of the vaporizer;
and wherein
the at least one electrical line of the vaporizer device is connected to the voltage source so that an electrical heating voltage can be applied to the vaporizer.
